(19) United States
(12) Patent Application Publication
(10) Pub. No.: US 2021/0007885 A1
(43) Pub. Date: Jan. 14, 2021

(54) POLYMERIC FILMS CONTAINING NANOPARTICLES ENDOWED WITH PHOTO-THERMAL EFFECT AND APPLICATION THEREOF AS THERMAL PATCHES

(71) Applicant: UNIVERSITA DEGLI STUDI Dl MILANO - BICOCCA, Milano (IT)

(72) Inventors: Giuseppe CHIRICO, Saronno (VA) (IT); Maddalena COLLINI, Milano (IT); Mykola BORZENKOV, Milano (IT); Piersandro PALLAVICINI, Pavia (IT)

(73) Assignee: UNIVERSITA DEGLI STUDI Dl MILANO - BICOCCA, Milano (IT)

(21) Appl. No.: 17/041,847

(22) PCT Filed: Mar. 27, 2019
(86) PCT No.: PCT/EP2019/057747

§ 371 Date: Sep. 25, 2020

(30) Foreign Application Priority Data
Mar. 28, 2018 (IT) 102018000004053

(51) Int. Cl. A61F 7/02; B32B 27/18; B32B 27/30; B32B 27/36; A61L 31/12

(52) U.S. Cl. CPC A61F 7/02; B32B 27/18; B82Y 40/00; B32B 27/365; A61L 31/128; B32B 27/306

(57) ABSTRACT

The present invention relates to thin polymeric films containing nanoparticles with tunable absorption in the visible and near infrared (NIR) region. When these films are irradiated with NIR sources, they show a pronounced photo-thermal effect. Said effect allows a localized temperature increase, which can be controlled both spatially and temporally. Once the irradiation source has been turned off, the temperature returns within a few seconds to the initial value and then raises again as soon as the film is irradiated again. These films can be used as reusable medical devices, with a controllable and reproducible heating profile, in particular thermal or heating patches.

### FIELD OF THE INVENTION

The present invention relates to the creation of polymeric films with highly efficient tunable and controllable photo-thermal effect that can be triggered with low excitation intensity over large surfaces and to the possibility to use them as a new class of medical devices (photo-thermal patches). The basic principle of this invention takes advantage of the optical properties of specific nanoparticles which are capable to convert (near infrared or visible) light into heat. This approach allows to obtain a rapid, controllable and repeatable local temperature increase. The developed technology, if applied for thermal patches, can lead to considerable advantages compared to existing chemically activated thermal patches: reusability, rapid, efficient and controllable thermal increase profile, absence of toxic and aggressive compounds, absence of side effects on patients of the compounds used for their fabrication.

### Background of the Invention

Musculoskeletal injury with medium- or long-term painful outcome is a common health problem worldwide. Non-treated sharp pain states may have serious long-term consequences: an appropriate treatment allows to prevent them to develop into chronic pain/suffering. Another very common and impairing form of muscular pain is muscular aching after physical activity: this is a common manifestation to those who start a new sport training program, but it can also happen to athletes who have intensified their training level.

The therapies usually performed comprise both pharmacological and non-pharmacological approaches. Among the non-pharmacological approaches, thermal therapy is broadly used. By thermal therapy it is meant any type of heat application to the body that allows to locally increase the temperature of the tissue. The physiological effects of thermal therapy include pain relief, increase of bloodstream and metabolism, and increase of the elasticity of connective tissue. This stimulates and promotes healing, mainly acting onto oxygen and nutrients supply. Moreover, a moderate increase in tissue temperature has a proven efficacy on the recovery of muscular performance, probably due to the modification of viscoelastic properties of the tissues.

Thermal therapy may be performed e.g. with thermal and electrical pads, or by means of deep-heating treatments (ultrasound and microwave diathermy); these treatments have the disadvantage that they require expensive devices and are provided by the specialized personnel. As an alternative to the above-mentioned methods, the chemically activated heating patches (thermal patches) are widely used thanks to their low cost and application ease. However, the existing thermal patches also have a number of disadvantages: heating rate is slow and uncontrolled, they can be used only once and may have unpleasant side effects (skin irritation and even burns).

Thermal therapy can be also obtained exploiting materials containing nanoparticles capable to release heat in response to EM irradiation in a given wavelength range; the photothermal nanoparticles can be incorporated within suitable supports for application to the human body (films, matrixes, patches. etc.); prior or during application to the body part requiring treatment, the support should be irradiated with light at a suitable wavelength and with a sufficient intensity so that the generated heat is released to the support and to the contacted body part. Examples of devices that could be used for photothermal of human body parts, are shown in: US2013/0310908, disclosing fibroin-based films for photothermal therapy including plasmonic nanoparticles mainly devoted to implantable electrical transducers applications; US2015/0086608 describes drug-loaded porous polymeric matrixes containing light-absorbing nanoparticles: upon irradiation, the nanoparticles generate heat which, in turn, promotes the release of the loaded drug. US2015/0209109 discloses bioadhesive matrices for tissue repair comprising an elastin-like polypeptide and a light-absorbing chromophore: the large heat generated by the chromophore is used to promote welding of adjacent disrupted tissue surfaces. US2015/0094518 discloses polymeric platforms for drug release: they contain an anticancer agent and, optionally, photothermically active nanoparticles. The publication Applied Surface Science, 435, 2018, pp. 1087-1095 describes the inkjet printing of copper sulfide nanoparticles onto a latex coated paper support, obtaining a film (thin layer of printed nanoparticles) suitable for the production of biomedical devices with photothermal effect. The construction of these biomedical devices entails a number of challenges: in particular, the uniform and quantitative incorporation of the desired amount of nanoparticles into the polymer structure is not easy to accomplish. The viscosity of the polymer compositions and the tendency of nanoparticles to aggregate, in fact, oppose to an efficient, uniform dispersion of the nanoparticles throughout the polymeric mass; as a consequence, the resulting products suffer from a non-homogeneous particle dispersion which translates into a reduced photothermal efficiency and non uniform heat release from the surface of the device, once irradiated. In order to ensure sufficient heat transfer from the device, manufacturers tend to increase the concentration of nanoparticles incorporated in the polymer and/or to increase the irradiation intensity: however these solutions are far from ideal in that they involve higher costs due to the use of larger amounts of nanoparticles and enhanced energy consumption for irradiating; moreover, the use of high intensity values can be harmful for the untreated portion of the skin if the irradiation area is not well controlled; finally, these approaches involve the risk of local overheating which may damage the concerned areas of the support and/or body areas of the patient exposed thereto. Therefore, none of the cited implementations of photothermal devices would allow a therapeutically relevant increase of the temperature over extended areas of the human skin (≅12×12 cm²) with safe doses of Near Infrared radiation. In addition, mentioned above patents do not provide with information about re-usability of fabricated devices

There is therefore still the need for new devices for thermal therapy (e.g. heating patches) which associate practicality of application to a better control of thermal profile, in favor of a treatment which is safer and easier to adapt to patient conditions. There is further the need to improve skin biocompatibility of the devices for thermal treatment, especially in case of treatments which require repeated applications. There is further the need for reusable devices, such as to allow for a less expensive treatment cycle compared to one based on the application of disposable patches. There is still finally the need for reusable devices, which provide performances which are reproducible and constant over time, without incurring a significant decrease.

### SUMMARY

The present invention relates to new thin polymeric films containing nanoparticles capable to release heat under irradiation (photo-thermal effect) with visible or near infrared (NIR) light, provided with an efficient, rapid, repeatable and controllable heating profile. Specifically, object of the invention is a polymeric film containing nanoparticles, said nanoparticles display a photo-thermal effect, which can be induced by light irradiation with wavelength between 0.4 μm and 1.2 μm, preferably between 0.5 μm and 1.0 μm, more preferably between 0.6 μm and 0.9 μm. In a particular embodiment, the invention concerns a selected combination of preferred nanoparticles in specific concentrations and supporting polymers (capable to form film), which achieves a highly uniform nanoparticle distribution, with consequent high efficiency of the photothermal effect and uniform heat response of the nanocomposite film; said combination also results in a device with enhanced thermal efficiency, expressed as amount of generated heat in respect of the applied radiation intensity; the high thermal efficiency allows to use irradiation intensities much lower than usually applied in the field of thermal therapy of similar purposes, with advantageous saving in energy costs and lessening the risks of high-intensity radiation, possibly harmful to the polymeric support and/or the exposed patient. According to this embodiment, one object of the invention is a polymeric film containing nanoparticles selected from the group consisting of Gold nanostars (GNS) and Prussian blue nanoparticles (PBNP), said nanoparticles being dispersed, as a whole at a concentration comprised between 0.005 and 0.1 nanoparticles/μm³ (preferably between 0.01 and 0.1 particles/μm³ or between 0.005 and 0.05 particles/μm³) in a film composition based on combination of polyvinyl alcohol with other polymers (e.g. PVP, sodium alginate, chitosan, hydroxypropyl methylcellulose) and with further cross-linking of the resulting combination. The films described herein provide a new class of medical devices for thermotherapy, in particular thermal patches, which can be activated with visible or near infrared (NIR) light radiation.

### DESCRIPTION OF THE DRAWINGS

FIG. 1. Photo-thermal effect obtained from the films of the present invention. When it is irradiated with visible or near infrared light, the film starts to absorb and to convert electromagnetic energy into heat. As soon as the source has been turned off, the heat is rapidly dissipated and the temperature returns to its initial value.

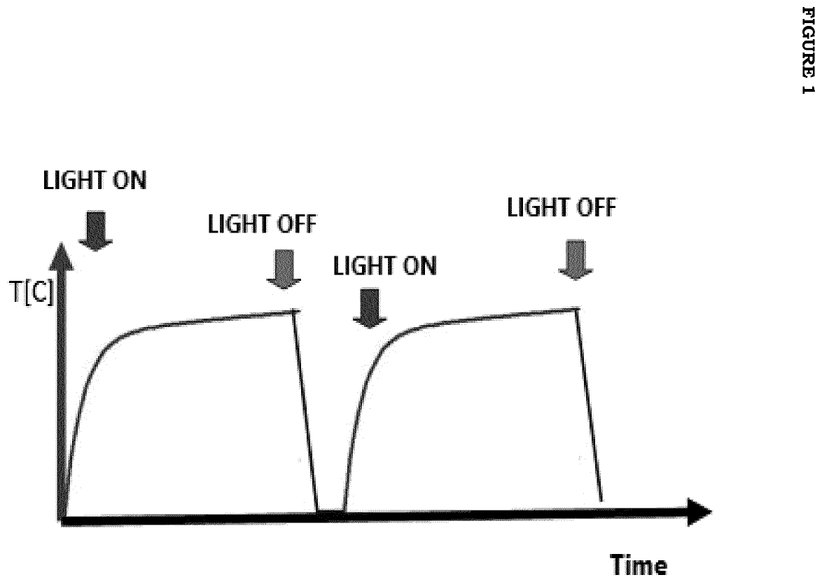

FIG. 2 (a). Spectrum of light extinction by an aqueous GNS solution (35-fold diluted stock solution); (b) Spectrum of light absorption by an aqueous PBNP solution (12-fold diluted stock solution).

FIG. 3. Photographs of the films containing nanoparticles The photograph on the left, panel A, refers to a film containing PBNP. In the photograph on the right, we show the visual comparison of the film without nanoparticles (panel B) and the film containing GNS (panel C).

FIG. 4: Images of the films obtained with reflection confocal microscopy. The images are projections of 50 planes of 37 μm×37 μm spaced 0.5 μm apart. Panel A: GNS film with 3% v/v concentration (150 μL in 5000 μL); Panel B film produced at 6% v/v concentration (300 μL in 5000 μL); Panel C: PBNP film: a film produced at 50% v/v concentration (2500 μL in 5000 μL)

FIG. 5. Increase in temperature of a 3% v/v GNS nanoparticles film from room temperature (20 Celsius degrees). Two irradiation cycles with NIR source are shown (film F1; irradiation power 80 mW; Irradiation intensity=0.16 W/cm²) In the panel on the right we show two exemplary images of the film portion which is irradiated with NIR light immediately after the beginning of irradiation and after 20 s of continuous irradiation. The temperature can be read from the temperature scale which is vertically placed.

FIG. 6: (a) First cycle of a series of 35 cycles of irradiation of a film F1 (irradiation intensity=0.16 W/cm²). (b) Last cycle of a series of 35 cycles of irradiation of a film F1 (irradiation intensity=0.16 W/cm²).

FIG. 7. Control of the stability of photo-thermal response of a film F1 under continued long-time irradiation (irradiation intensity=0.16 W/cm²). The saturation value of temperature is 28±2° C., it does not show any considerable decrease over time starting from an irradiation time equal to 10 s. The dashed line is a fit of the data onto a logistic curve of the type f(t)=T_{∞}+(T₀−T_{∞})/(1+(t/τ)^{p}). The best-fit values are: T₀=20.4±0.04; T_{∞}=28.2±0.002; τ=5.9±0.04; p=2.5±0.02.

FIG. 8. Exemplary curves of the temperature increase induced by continuous irradiation with NIR radiation on 6% v/v GNS films (F2 and F4, see Tables 1,2,3): irradiation power=80 mW (1=0.16 W/cm², lower curve) and 100 mW (I=0.2 W/cm², upper curve). The data were analyzed with biexponential increase curves (dashed curves). Increase times are τ₁=4.4±0.03 s and τ₂=29.8±0.2 s for I=0.16 W/cm² and τ₁=4.5±0.04 s and τ₂=34.0±0.5 s I=0.2 W/cm².

FIG. 9. Photo-thermal effect (global temperature increase under continuous irradiation) on films produced with GNS nanoparticles, versus irradiation intensity (squares, films obtained with a volume dilution equal to 3% v/v; circles, films obtained with a volume dilution equal to 6% v/v). The dashed lines are obtained by best-fitting the data to direct proportionality lines with slopes of 66±3 [° C. cm²/W] and 104±4 [° C. cm²/W], respectively for the two films. The ratio of the two slopes is 1.6±0.07, in reasonable accordance with the expected ratio of 2.

FIG. 10: Panel A: photo-thermal kinetics on a film containing PBNPs (formulation F5) under effect of pulsed irradiation with infrared radiation (0.80 μm, intensity 0.16 W/cm²). Two activation and relaxation cycles are shown. Panels B and C show the details of activation (B) and relaxation (C) kinetics. The solid curves are the exponential fits to the data and correspond to the time of 5.8±0.5 for activation and 8±0.5 s for relaxation.

FIG. 11. Dependence of the photo-thermal effect on the irradiation power (circles, wavelength=0.80 μm; squares, wavelength=0.7 μm) onto a PBNP film (formulation F5). The dashed curves are linear fits to the data and correspond to slopes ΔT/ΔI=160±4 [° C. cm²/W] (for 0.7 μm) and ΔT/ΔI=136±4 [° C. cm²/W] (for 0.8 μm). The sample was obtained by diluting the stock solution to 50% V/V.

FIG. 12: Outline of the assessment of photo-thermal efficiency on porcine skin with a source at wavelength 0.80 μm on a film of formulation F2 with 6% v/v GNS nanoparticles.

FIG. 13. Thermal image of the temperature increase measured on the tip of a finger of one of the inventors. The film (formulation F2) was placed onto the skin and wrapped so as to allow adhesion to the body. The temperature measured at the center of the irradiated zone is 39° C., equal to an increase of about 4 Celsius degrees.

FIG. 14. Photograph of a single LED matrix used in an embodiment of the invention.

FIG. 15. Emission profile of the photodiode without collimation lens measured at 60 cm distance.

FIG. 16. (A) The right panel reports the scheme of the LED source box and the irradiation (red square) area. The left panel reports the details of the LED source box; (B): Optical sketch of the Koheler illumination setup that is implemented in the LED source box; (C) drawing of the optical path of the rays in the Koheler illumination setup that shows that the illumination field at the patient position is the pupil of the field lens magnified by the collection lens.

FIG. 17. Sketch of the position of the sampling points on the tyre thin slab, on which the temperature was measured.

FIG. 18. Heating profile under irradiation with LED of patch: the concentration of starting reagents was 10 mM; the current driving the LEDs was 0.99 A, the irradiation area was 8×8 cm². Solid red line (left to “off”) is a fit of heating profile (τ₁=6.4 s and τ₂=32.1 s); solid blue line (right to “off”) is a fit of the cooling profile (τ₁=24 s; τ₂=10.6 s).

### DETAILED DESCRIPTION OF THE INVENTION

The term “film” used herein in relation to the invention in all its embodiments, identifies a thin laminar structure, suitable to be applied to a portion of patient's skin, substantially adapting to the curvature thereof. The film can be of monolayer or multilayer type. It can have adhesive properties to skin (e.g. by including adhesive polymers); alternatively, it does not have adhesive properties but it is provided, totally or partially, on the side intended to contact the patient's skin, with appropriate adhesive areas obtained by application of a further layer of adhesive material; each adhesive area is preferably covered by an appropriate protective layer which can be removed upon use. In a further variation, the film does not have adhesive properties to skin and is not provided with adhesive areas: in this case it carries out its function being only placed onto the skin area of interest, optionally held on the spot by way of separate structures (elastic tapes, bandages, patches, etc.).

The term “thin” referred to the film of the present invention in all its embodiments, is broadly meant to include film thicknesses between 30 and 200 μm, preferably between 70 and 160 μm, more preferably between 80 and 120 μm, e.g. 100 or 110 μm. The film with such thicknesses can be used as such as thermal patch, or it can be provided with a support (backing) to increase its consistency/capability of being handled; the possible support must be transparent to irradiation, at least in the specific wavelength which is effectively applied, so as to allow the photo-thermal effect to establish inside the film. The film and the possible support may have variable shape and size, depending on the specific areas of the human or animal body to be treated: as an alternative to the common standard shapes such as the rectangular, circular or ovoid, it is possible for example to prepare it as a glove or sock (for application to hands or feet), or tubular (for application to a limb), etc.

Regarding the nature of the polymer in the film, each non-toxic polymer compatible with human and/or animal skin can be in principle used. Among them it is possible to mention as examples: polysaccharides (e.g. alginate, xanthan, carrageenan, hyaluronan, pectin, chitosan, cellulose), polylactides, polyacrylates, polymethacrylates, polyoleolefins, polyvinyl polymers (e.g. polyvinyl alcohol or polyvinylpyrrolidone), polyurethanes, polyamides, polyimides, polyethers, polyesters, polyacetates, polycarbonates, rubbers, polysiloxanes, and derivatives thereof (e.g. cross-linked derivatives) and mixtures thereof. Preferred polymers according to the invention are polyvinyl alcohol, polyvinylpyrrolidone and/or chitosan, sodium alginate and hydroxypropyl methylcellulose and the corresponding cross-linked derivatives; the biocompatibility of the above mentioned polymers is well known, as reported in for example: http://www.inchem.org/documents/jecfa/jecmono/v52je09.htm, https://doi.org/10.1177/109158189801700408 and http://pubs.rsc.org/en/content/articlelanding/2015/tx/c4tx00102h#!divAbstract. In a most preferred embodiment, particularly suited to optimize the uniformity of nanoparticle distribution within the film and the thermal efficiency of the film, the film comprises cross-linked polyvinyl alcohol: according to this embodiment, the Gold nanostars (GNS) or Prussian blue nanoparticles (PBNP) are dispersed, as a whole at a concentration comprised between 0.005 and 0.1 nanoparticles/μm³, in a film composition based on PVA (with possible other polymers), where the resulting composition is subjected to cross-linking; preferably, the cross-linked polyvinyl alcohol represents at least 40% by weight of the total amount of polymers making up the film; alternatively, when referred to the composition of the film prior to cross-linking, polyvinyl alcohol represents at least 40% by weight of the total amount of polymers in the composition to be subjected to cross-linking.

The term “nanoparticles” used herein in relation to the invention in all its embodiments, identifies particles of nanometric size, preferably less than 100 nm (e.g. between 5 and 75 nm, or between 5 and 50 nm or between 5 and 30 nm). All types of nanoparticles which show a photo-thermal effect following irradiation with visible (0.4 μm-0.7 μm) or near infrared (0.7 μm-1.2 μm) light are suitable for this invention. Particularly advantageous results are obtained when using nanoparticles which further have an efficiency of conversion between absorbed radiation and emitted heat (herein also measured as Specific Adsorption Rate) higher than 50 kW/g, particularly between 50 and 300 kW/g, preferably between 150 and 300 kW/g. The Specific Adsorption Rate (conventionally referred to as SAR) is defined as:

$$S A R = \frac{C}{{M}_{NP}} \frac{d Δ T}{dt} {\vert}_{t = 0}$$

wherein C is the thermal capacity of the suspension and M_{NP} is the total mass of the nanoparticles. Finally, nanoparticles with low toxicity and surface properties suitable for their homogeneous dispersion in the polymeric matrix are preferred.

Preferred examples of nanoparticles satisfying said requirements are gold nanoparticles, in particular Gold Nanostars (herein abbreviated as GNS) and Prussian blue nanoparticles (herein abbreviated as PBNP).

GNSs are commercially available e.g. from NanoSeedz and NanoimmunoTech (https://www.nanoimmunotech.eu/en/Shop/-/Gold-NanoStars and https://www.nanoseedz.com/Au_nanostar.html). GNSs and PBNPs are biocompatible and nontoxic; PBNPs are also approved by the U.S. Food and Drug Administration (FDA).

GNSs can be obtained by known procedures, which include using the surfactant Triton X-100 (see e.g. Pallavicini et al., Chem. Commun., 2013, 49, 6265-6276, herein incorporated by reference). Said procedures allow to precisely regulate the position of the plasmon resonance peak(s) in the NIR range (surfactant type, reagent concentration),In particular, GNSs show two or more localized surface plasmon resonances (LSPR, characterized by two intense peaks in the range 0.6-0.9 μm e 1.1-1.6 μm), which induce a thermal relaxation (=heat release) when the GNSs are irradiated.

Also the PBNPs can be obtained by means of known procedures (see, e.g., e.g. Supramolecular Chemistry, 2017, 19, 1-11, herein incorporated by reference): it envisages the reaction of FeCl³⁺ with citric acid and the subsequent addition, to the reaction mixture, of a solution of K4[Fe(CN)6] and citric acid. PBNPs show an intense absorption band with a maximum at around 0.7 μm. The irradiation in this band results in a thermal relaxation corresponding to heat release.

The photo-thermal effect of the present films is consequent to the application of the irradiation. Irradiation can be supplied by any suitable device emitting visible and/or NIR light in the above stated wavelength ranges. Advantageously, when the nanoparticles are stable in the chosen polymers solutions and uniformly distributed in the resulted cross-linked films as a result of the high thermal efficiency of the present compositions, particularly when the film comprises cross-linked polyvinyl alcohol, the irradiation can be performed with intensities considerably lower than those commonly applied in this field: in fact, as shown in the examples, levels of heat generation optimally suited for thermal treatments were obtained with irradiation intensities around 0.2 W/cm², for polymeric films containing the present nanoparticles at concentrations in the order of 0.010-0.030 nanoparticles/μm³. Therefore, in a typical embodiment, the invention concerns the use of a heat-releasing medical patch comprising a film as above described, for use in thermal therapy in humans or animals, wherein the heat release is obtained by using irradiation intensities lower than 10 W/cm², or lower than 5 W/cm² or even lower than 1 W/cm²; preferably, the film in this embodiment comprises cross-linked polyvinyl alcohol, as described above.

For irradiating purposes, any irradiation device emitting light (light source) in the visible or NIR range, can be employed for the purpose of the invention; examples of standard irradiation devices are mentioned in the experimental examples 4 and 5. Special irradiating devices, preferred although not indispensable to obtain the effects of the present invention, are LED-based ones, as described in the experimental example 6: among them, particularly interesting are those equipped with optical systems enabling to direct and change the shape of the irradiation area to suit any particular need for therapy: for example those employing Fresnel acrylic lenses and/or Koheler illumination optics (see example 6).

The films of the present invention may release heat repeatedly and reproducibly for an extended number of times, depending on the number of irradiations applied: in experimental testing, up to 40 heating cycles were applied to the films of the invention, obtaining a substantially constant response, i.e. with a loss of the maximum temperature reached by the film below 1%. A broad reuse of the same films is thus possible, with an obvious advantage compared to thermotherapy devices (chemically activated heating patches and plasters) based on exothermic chemical reactions, which definitely exhaust and have to be disposed after a single use.

As a further advantage, repeatedly using the films according to the invention does not involves substantial modifications of structure/functionality of the film. For example, the nanoparticles as GNSs and PBNPs guarantee a constant (in intensity and response time) photo-thermal effect following repeated use, i.e. after 40 or more uses. Said stability/reproducibility of response is a particularly important requirement, since it guarantees that the present films can be “effectively” reused, i. e. with the necessary precision and safety. The films retain their photothermal efficiency even after 2 months of storage at room temperature and humidity, confirming the film stability and NP stability within the film structure.

Moreover, the film with nanoparticles such as GNSs or PBNPs, due their high SAR values, have the further advantage of a particularly short induction time (onset of the photo-thermal response), i.e. reaching the desired temperature typically within 5 s from the beginning of irradiation. This is particularly evident for the film compositions in accordance with the aforementioned preferred embodiment, in which GNS or PBNP are dispersed at the aforementioned concentration ranges in a film composition comprising cross-linked polyvinyl alcohol. Said aspect is highly interesting for applications, considering that traditional devices based on exothermic chemical reactions or electro-heated devices have a much longer induction time to reach desired temperature. The same GNS and PBNP nanoparticles, preferably formulated in accordance with the aforementioned preferred embodiment, result in films with short times of termination of photo-thermal effect, typically within about 10 seconds from the end of irradiation: this characteristic allows a precise control of the effect within a specific time window, which is easy to be assessed based on the duration of the irradiation.

Finally, the above-mentioned films of GNSs and PBNPs also have the further advantage to rapidly reach a plateau of constant temperature, which lasts during the whole irradiation time: this avoids undesired overheating phenomena which could damage the patient and/or the device, and spares the necessity to monitor/adjust the irradiation intensity during treatment.

Therefore, in addition to the general advantage provided by the system as a whole, the use of GNSs and/or PBNPs or other nanoparticles guarantees a special versatility/practicality of application of the film in the thermotherapeutic field, e.g. in the form of heating plasters.

The present nanoparticles are dispersed in the film (or in part thereof) at such a concentration to produce, following irradiation, a significant thermal effect that can be exploited for thermal therapy; preferably, for said purpose, nanoparticles concentrations between 0.005 and 0.1 particles/μm³, preferably between 0.01 and 0.1 particles/μm³ or between 0.005 and 0.05 particles/may be used. The term “or part thereof” used herein with reference to the present film in all its embodiments identifies the photo-thermally active part of the film: it can correspond to the whole film or to one or more selected parts thereof where it is desired to generate heat: in particular, the film can contain photo-thermally active areas conveniently placed such as that, after application onto the patient, they develop heat at specific body areas requiring the thermotherapeutic effect. The above-mentioned concentration values are therefore meant as referred to the photo-thermally active area of the film, which can be the whole film or one or more parts thereof.

Besides the nanoparticles, the film can contain further ingredients which are commonly used in the preparation of films suitable for application onto the skin: among them can be mentioned: plasticizers (e.g. polyethylene glycol 200, diethylene glycol, propylene glycol, glycerol, etc.), preservatives, possible active ingredients useful for topical administration (e.g. anti-inflammatory agents, painkillers, moisturizers, etc.), bioadhesive substances, etc.

For the purposes of the preparation of the present films, it is in principle possible to use any process which allows a homogeneous dispersion of the nanoparticles (and further ingredients) within the selected polymer. For example, it is possible to incorporate said nanoparticles and excipients in the step of polymer formation, i.e. by including them in the mixture consisting of the relative precursors (monomers and possible polymerization catalysts); preferably, the suspension containing said nanoparticles is added to a solution of said polymer or precursor thereof, forming a nanocomposite film; alternatively it is possible to start with an already formed polymer (for example at the fluid state) and disperse the nanoparticles and said excipients in the aqueous solutions of the selected polymers. The incorporation of the particles and said other ingredients is also possible in an intermediate step of formation of the polymeric matrix, for example after formation of the polymer, but before its cross-linking. A preferred preparation process concerns the cross-linking step of the polymer(s) used during the film preparation stage or when the film is formed. In particular the polyvinyl alcohol was crosslinked in the present film. Said cross-linking provides a further contribution to immobilizing the nanoparticles, preventing their aggregation, nanoparticles release and/or leaking during manufacturing and/or during the service life of the film, thus contributing to the efficiency and stability of thermal response of the film. In addition, cross-linking improves in general the film stability and resistance as non-cross-linked films based on chosen polymers tend to dissolve when soaked in water. As said, the cross-linking can be obtained by adding to the polymer an appropriate cross-linking agent, e.g. citric acid or other cross-linking agent selected depending on the specific chosen polymer. The choice of citric acid, while not indispensable for the purposes of the invention, is preferred in that it represents a “green”, eco-compatible, highly skin-tolerable cross-linking agent in comparison with widely used but toxic glutaraldehyde. Non-chemical, for example physical cross-linking can be also applied. In addition or in alternative, the nanoparticles bearing functional groups on their surfaces (e.g. carboxylic COOH) can act as additional cross-linking centers.

The incorporation of the nanoparticles to the polymer or precursor thereof preferably occurs by adding, to said polymer or precursor, nanoparticles in the form of suspension in an appropriate solvent, preferably aqueous suspension. If GNSs or similar nanoparticles are used, the above-mentioned process can advantageously include a pegylating (coating of the nanoparticles with a suitable polyethylene glycol, e.g. PEG 5000 containing a thiol group for binding with gold) prior incorporation into polymeric solution. Such treatment further improves the stability of GNSs in aqueous solutions and their dispersibility. Moreover, this step of pegylating allows to remove most of the toxic surfactants used for synthesis, which can give biocompatibility problems. The process of film preparation further comprises a step of deposition of the final product in laminar form, so as to form a film.

The invention is now described by way of the following non-limiting experimental examples.

### EXPERIMENTAL PART

### Example 1 GNSs Synthesis

GNSs were synthesized by “seed-growth” technique in the presence of the nonionic surfactant Triton X-100, as previously reported (Pallavicini, 2013 op.cit.).

All the glassware used for production and subsequent covering was always pre-treated with aqua regia before use.

5 mL of 5*10⁻⁴M HAuCl₄ in water are added to 5 mL of an aqueous TritonX-100 solution. Then, 0.6 mL of a pre-ice cooled (0.01M) NaBH₄ solution in water are added. The mixture is mildly mixed by hand and a reddish-brown color appears. The stock solution is then kept in ice and used within a few hours.

The growth solution is prepared in 20 mL vials. 250 μL (0.004M) AgNO₃ in water and 5 mL (0.001M) HAuCl₄ in water, in this sequence, are added to a 5 mL of an aqueous (0.2M) Triton X-100 solution. Then, 140-400 μL of an aqueous solution of ascorbic acid (0.0788M) are added. The solution, after a gentle blending, becomes colorless. Immediately afterwards, 12 μL stock solution are added. The samples are left to equilibrate for 1 hour at room temperature.

The GNSs thereby obtained are preferably coated with polyethylene glycol containing a —SH group, for example SH-PEG₅₀₀₀-OCH₃ or SH-PEG₅₀₀₀-COOH. Pegylation is obtained by simultaneously adding 200 μL of an aqueous solution of 10⁻³ M PEG-thiols to 10 mL of a GNS solution prepared as described above, reaching a final concentration of 20 μM PEG-thiols. The solution obtained is left to equilibrate for three hours at room temperature under the action of a gentle blending by shaker with subsequent ultracentrifugation (3 times, 25 min, 13000 rpm).

In order to obtain an enhanced photo-thermal effect, concentrated GNS solutions were prepared, using high volumes (100 mL) of GNSs in the process of pegylation and re-dissolving the GNS sediment after the last ultracentrifugation cycle in 1 mL double-distilled water. In this way 100-fold concentrated (≈6 mg Au/mL) solutions are obtained. In case of coating with SH-PEG₅₀₀₀-COOH of the solution, the final pH is adjusted at about pH=8 by adding NaOH (0.05 M solution).

### Example 2 PBNSs Synthesis

PBNPs were synthesized according to the protocol shown in Supramolecular Chemistry, 2017, 19, 1-11.100 ml of a solution of 1.0 mM FeCl³⁺ and of 0.025 M citric acid are heated to 60° C., while continuously blending. A second solution (1.0 mM K4[Fe(CN)6] containing the same citric acid concentration is heated to 60° C. and added to the Fe³⁺ solution, obtaining an intense blue color. After 1 minute of blending at 60° C., the solution is left to cool at room temperature. The sediment of centrifuged PB nanoparticles is resuspended in half the original volume. The concentration of the nanoparticles in the final solution can be increased by at least a factor 10 by increasing from 1 mM to 10 mM the concentrations of the starting Fe^{III} (as FeCl₃) and Fe^{II} (as K₄[FeCN)₆]) reagents.

### Example 3 Preparation of the Films

In order to form the polymeric films, the following polymers were used: polyvinyl alcohol, PVA (with degree of saponification higher than 70%); polyvinyl pyrrolidone, PVP (PM 50000); (medium and low molecular weight) chitosan. PVA shows a wide range of useful properties, such as low toxicity, biocompatibility, hydrophilicity, chemical stability and excellent film-forming capabilities. PVP is broadly used and has been approved by the FDA for different uses as coating agent, polymeric membranes and material for the controlled drug release. Chitosan is odorless, biocompatible, biodegradable and nontoxic. In particular, PVA allows the formation of hydrogen bonds between OH and NH₂ groups. Mixtures of the above-mentioned polymers were used herein, in order to optimize the properties of the polymeric films obtained.

The polyethylene glycol PEG-200 (11% by weight of the total weight of the polymer) is used in this step only as plasticizer.

In order to increase the resistance of the films and their mechanical properties, cross-linking is performed. Citric acid (11% by weight in relation to PVA weight), which is nontoxic and approved by FDA, is selected as cross-linking agent. No other mineral acid (e.g. HCl) is used in the procedure as a catalyst, since the process is promoted by way of thermal sintering of the formed films (130° C.; sintering time 10-30 min).

The six formulations (hereinafter referred to as F #) of films were produced with different polymers and different polymer ratios, also incorporating different types of nanoparticles and in different amount, as described in more detail in the following tables and preparation procedures.

TABLE 1
Formulations of films F1-F4 (GNS)

| | | | | | | | Concen- |
|---|---|---|---|---|---|---|---|
| | | | | | | Pegylated | tration |
| | | | | | Citric | GNS | of the |
| Compo- | H₂O, | PVA, | PVP, | PEG, | acid, | solution, | Solution |
| sition | ml | g | g | g | g | μl | V/V % |
| F1 | 4.85 | 0.31 | 0.25 | 0.0616 | 0.0341 | 150 | 3 |
| F2 | 4.7 | 0.31 | 0.25 | 0.0616 | 0.0341 | 300 | 6 |
| F3 | 4.85 | 0.375 | 0.25 | 0.068 | 0.041 | 150 | 3 |
| F4 | 4.7 | 0.375 | 0.25 | 0.068 | 0.041 | 300 | 6 |

N.B. Total volume of the solution before drying = 5000 μL. For pegylating, PEG-200 was used.

To produce formulations F1-F4, known amounts of PVP and PVA were mixed with water and kept 1 hour at 90° C. until complete polymer dissolution. Then, the plasticizing agent (11% by weight) and a given volume of the GNS solution are added and the mixture is stirred for 5 hours at 40° C. Citric acid (11% of the weight of PVA) is added and the solution is further stirred for 1 hour at 40° C. The mixture is poured in a Petri dish. Once the film has formed, it is placed in heater (130° C. 20 min) to complete the cross-linking process.

TABLE 2
Formulation of film F5 (PBNP)

| | | | | | | | Concen- |
|---|---|---|---|---|---|---|---|
| | | | | | | | tration |
| | | | | | Citric | PBNP | of the |
| Compo- | H₂O, | PVA, | PVP, | PEG, | acid, | solution, | solution |
| sition | ml | g | g | g | g | ml | V/V % |
| F5 | 2.5 | 0.375 | 0.25 | 0.068 | 0.041 | 2.5 | 50 |

N.B. Total volume of the solution before drying = 5000 μL. For pegylating, PEG-200 was used. The concentration of starting reagents was here 1 mM.

To produce the formulation F5, known amounts of PVP and PVA are mixed with (2.5 ml) water and kept 1.5 hour at 90° C. until complete polymer dissolution. The plasticizing agent (PEG 200) and 2.5 ml of PBNP solution are added and the mixture is stirred for 5 h at 40° C. Citric acid is then added and the solution is further stirred for 1 hour at 40° C. The mixture is poured in a Petri dish. Once the film has formed, it is placed in heater (130° C. 20 min) to complete the cross-linking process.

TABLE 3
Formulation of film F6 (GNS)

| | | | 2% (w/v) | | | | Concen- |
|---|---|---|---|---|---|---|---|
| | | | chitosan | | | Pegylated | tration |
| | | | in 2% | | Citric | GNS | of the |
| Compo- | H₂O, | PVA, | acetic | PEG, | acid, | solution, | solution |
| sition | ml | g | acid, ml | g | g | μl | V/V % |
| F6 | 2.2 | 0.375 | 2.5 | 0.041 | 0.041 | 300 | 6 |

N.B. Total volume of the solution before drying = 5000 μL. For pegylating, PEG-200 was used.

To produce the formulation F6, PVA is dissolved in 2.2 ml water and kept 1.5 hours at 90° C. until complete polymer dissolution. The plasticizing agent (PEG 200) and 2.5 ml of chitosan solution are then added and the mixture is stirred for 1 h at 40° C. The GNS solution is added and the mixture is stirred for 5 hours at 40° C. Then citric acid is added and the mixture is further stirred for 1 hour at 40° C. and poured in a Petri dish. Once the film has formed, it is placed in heater (130° C. 20 min) to complete the cross-linking process.

### Example 4 Properties of the Produced Films

### 4.1 Transparency/Color

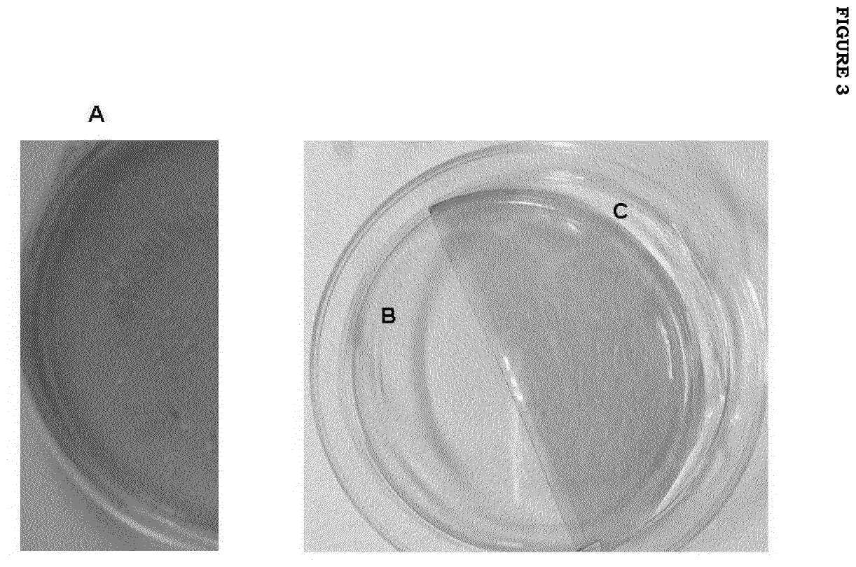

The inclusion of nanoparticles in the films influences their transparency in the visible range: the films become semitransparent with colors ranging from blue (GNSs) to dark blue (PBNPs). The appearance of these films is reported as reference in the photographs of FIG. 3.

### 4.2 Distribution/Concentration

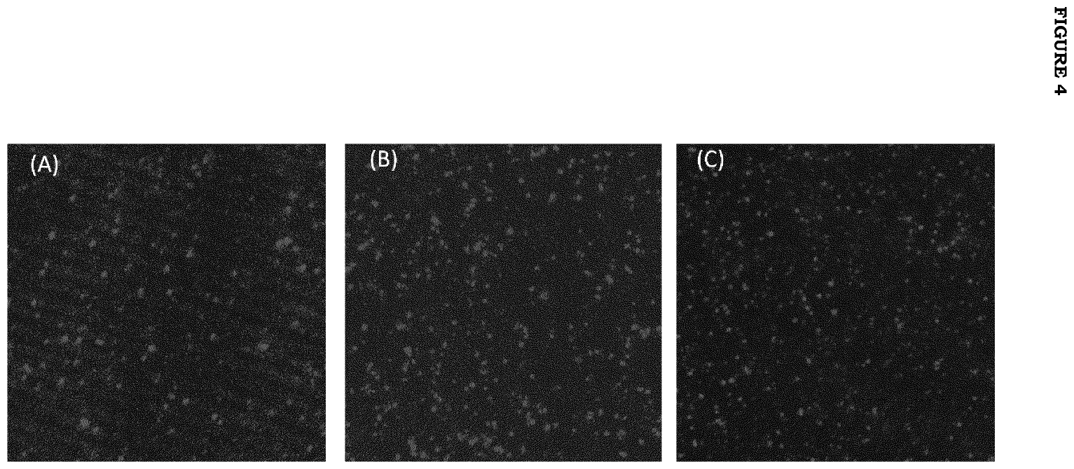

The films were also studied at the scanning optical microscope (confocal, reflection-mode, FIG. 4). The study showed that the nanoparticles distribution is uniform in the polymeric matrix. The particles appear as low-resolution spots in the images. By acquiring images at different heights (z-stack), it is possible to obtain their volumetric distribution, from which we could measure the effective concentration of nanoparticles in the produced films.

Analysis by reflection confocal microscopy allows to assess the effective concentration of nanoparticles in the films at the end of the production. Planes at different heights corresponding to a given volume calculated as the width of the visual field multiplied by the number of plans and their spacing. On each plane, the spots which have a size equal to the optical resolution of the microscope are assessed (the nanoparticles are in fact under-resolved with size of about 0.3 μm). The persistence of the spot along the z axis (optical axis of the microscope) is of about 8 planes±1 (spaced 0.5 μm). We obtain the value of the number of nanoparticles in the volume examined under the microscope by counting all the spots and dividing this number by the number of persistence planes. From this analysis we verify that for the samples at 3% v/v and 6% v/v of 100×GNS stock solution, the density changes by a factor 2 within the experimental errors.

The values found for the two preparations in FIGS. 4A and 4B are C=0.015±0.002 np/μm³ and 0.028±0.004 np/μm³ and C=0.032±0.002 np/μm³ for the sample with BPNP (FIG. 4C. Since the nanoparticles have sizes of the order of 20-30 nm, we estimate that also the fraction of film volume occupied by the nanoparticles is of only 2×10⁻⁵%-5×10⁻⁵%.

### 4.3 Folding Endurance

We cut a square strip of 4 cm² area of the films produced as described (prepared with both the nanoparticles types) and bent at 90 degrees and extended again for a number of times until breaking the film. The number of bendings necessary for breaking is considered as a measure of resistance to bending (see Table 4).

TABLE 4
Resistance to bending of films with nanoparticles

| Composition | Resistance to bending |
|---|---|
| F1-F2 | 240 |
| F2-F4 | >260 |
| F5 | >260 |
| F6 | >260 |

### 4.4 Thickness of the Film.

Using the “solvent casting” method, we obtained a highly reproducible thickness (±15%) of film equal to: thickness=110 (±15) μm.

### 4.5 Photo-Thermal Properties

We studied the photo-thermal properties of the nanoparticle films activated by irradiation with radiations having wavelength of 0.80 μm and 0.71 μm (source: Ti:Sapphire laser, pulse repetition 80 MHz, pulse width 200 fs on the sample. Tsunami and MaiTai Models, Spectra Physics, CA, USA. Minimum spectral range 690 nm-960 nm). The laser beam was focused with a single plan-convex lens and the sample was set at a distance at which the spot size was about 7-10 mm in diameter. The wavelengths are selected in accordance with the maximal absorption resonance of the surface plasmons, LSPR. The two wavelengths used herein satisfy this requirement for gold nanoparticles (GNSs) and Prussian blue nanoparticles (PBNPs). During irradiation, we registered temperature changes of the films by means of a thermo-camera (FLIR, E40, USA) and analyzed the videos by means of a support software of the same manufacturer.

### Photo-Thermal Effect of the Films Containing GNSs

The photo-thermal effect was assessed on two series of samples at nanoparticles concentrations of C=0.015±0.002 np/μm³ and 0.028±0.004 np/μm³, irradiated with NIR radiation at the wavelength of 0.8 μm. For all the prepared films we measured a rapid increase of temperature flattening within about 20 s at a level which depends on the irradiation power. As a control, the identical irradiation of films with the same polymer composition, free of nanoparticles, showed non-significant temperature increases, which are within the variability of measurement of the thermo-camera (+/−0.1° C.).

The temperature increase obtained from the films loaded with nanoparticles is higher than that obtained from suspensions of similar concentration. This fact can be explained by the reduced thermal conductivity (mainly with air) when using films, compared to that (of water) when using suspensions.

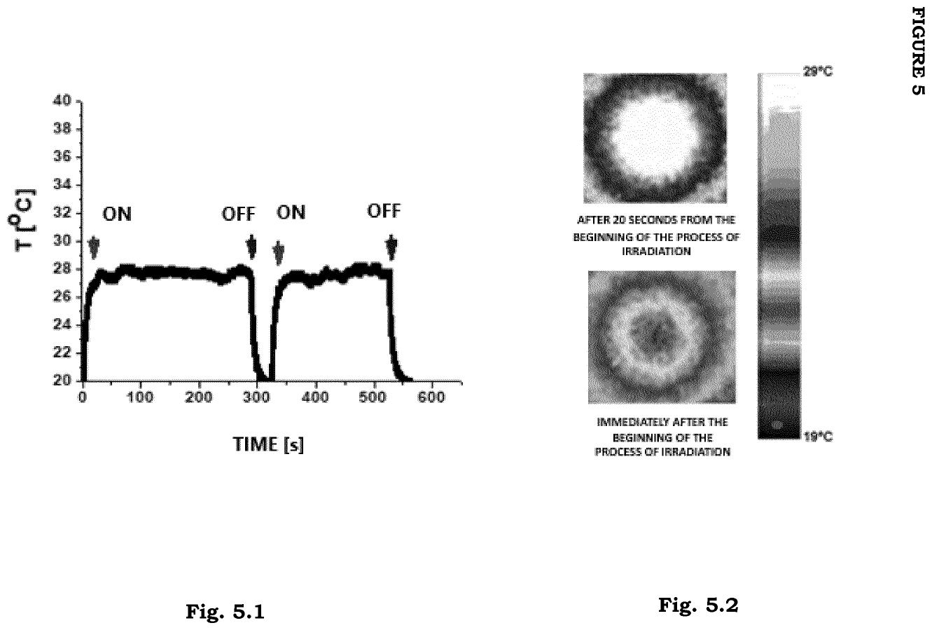

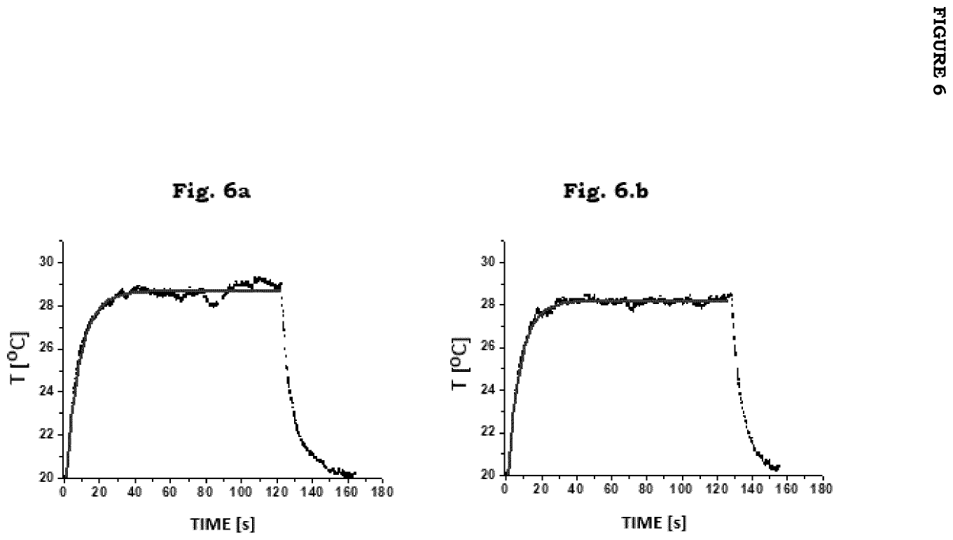

In the tested films, the film temperature returns within room temperature in less than 5 s after NIR irradiation has been interrupted. Heating and cooling cycle can be repeated (FIG. 5) a very high number of times without considerably losing photo-thermal efficiency of the film. This is shown in FIG. 6 where the first and thirty-fifth cycles of thermal activation and quenching of a film F1 are reported. As it can be noted, no degradation of the photo-thermal efficiency is measurable at least for a number of cycles equal to 35. As a further control of the photo-thermal effect of the prepared films, we applied continuous irradiation to a film F1 for 17 minutes, without detecting any considerable loss of efficiency.

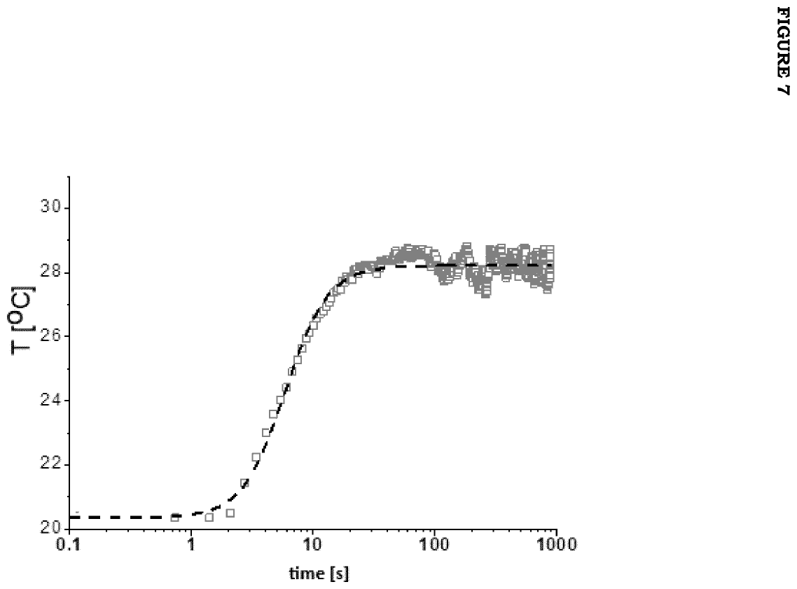

FIG. 5 demonstrates that the films can be used to induce localized heating, efficiently activated by NIR or visible light, with rapid response and high stability for a continuous and repeated use (FIGS. 6, 7). This allows to envisage applications with tailored, easy-to-plan heating profiles.

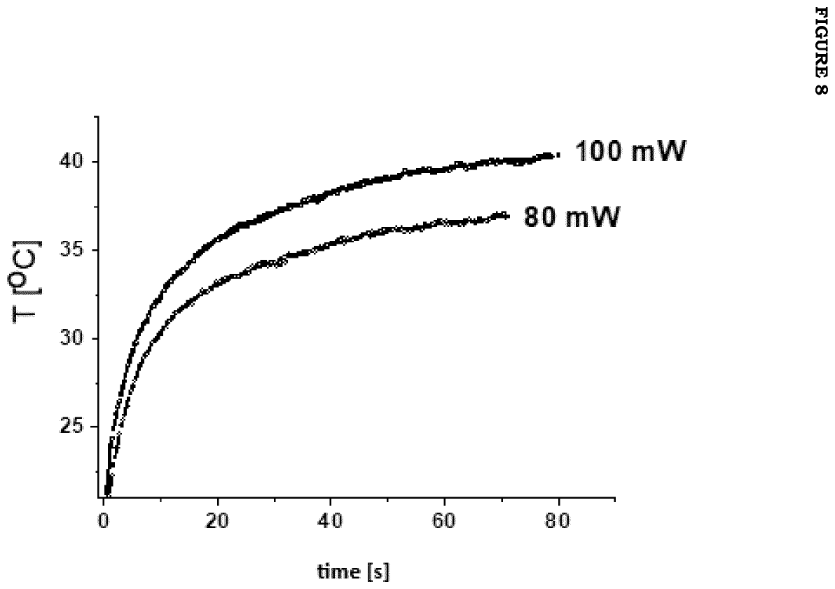

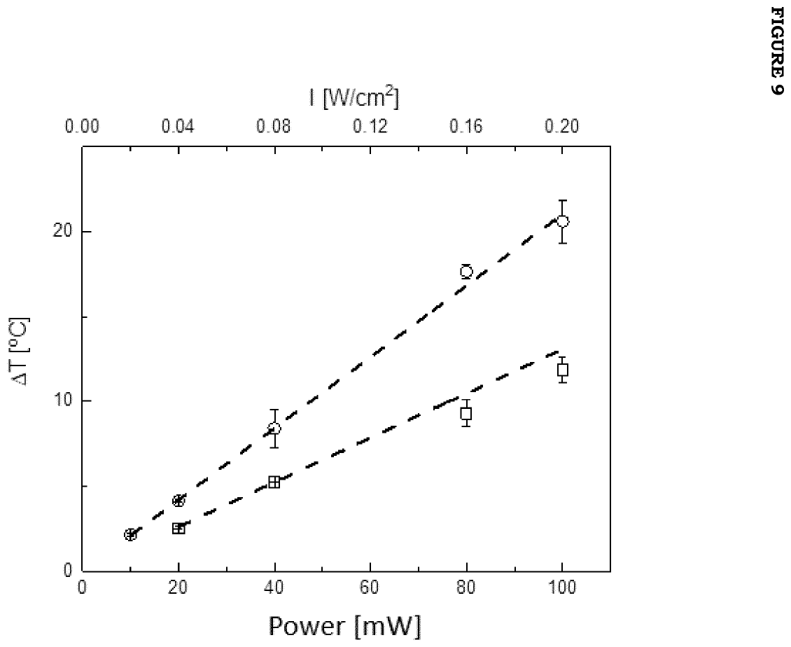

The temperature increase generally depends on nanoparticles concentration (linearly), on irradiation time (with an initially linear increase and the reaching of a plateau level for times >10 s, FIG. 8) and on the irradiation intensity (linearly, FIG. 9). The increase of temperature over time, during a continuous irradiation of the film, is well described by a bi-exponential increase curve (FIG. 8). The shorter time is related to the absorption of NIR radiation by the nanoparticles and to heat diffusion inside the irradiation spot. The longer time is related to the exchange with the environment (the laboratory or the tissue/body with which it is contacted). In any case, the highest temperature increase was observed for films prepared at nanoparticles concentrations C≅=0.03 np/μm³, and such increase changes linearly with the concentration at least up to concentrations equal to C≅0.03 np/μm³.

The range of temperature increase depends linearly on the irradiation intensity, as shown in FIG. 9.

### Photo-Thermal Effect of the Films Containing PBNPs

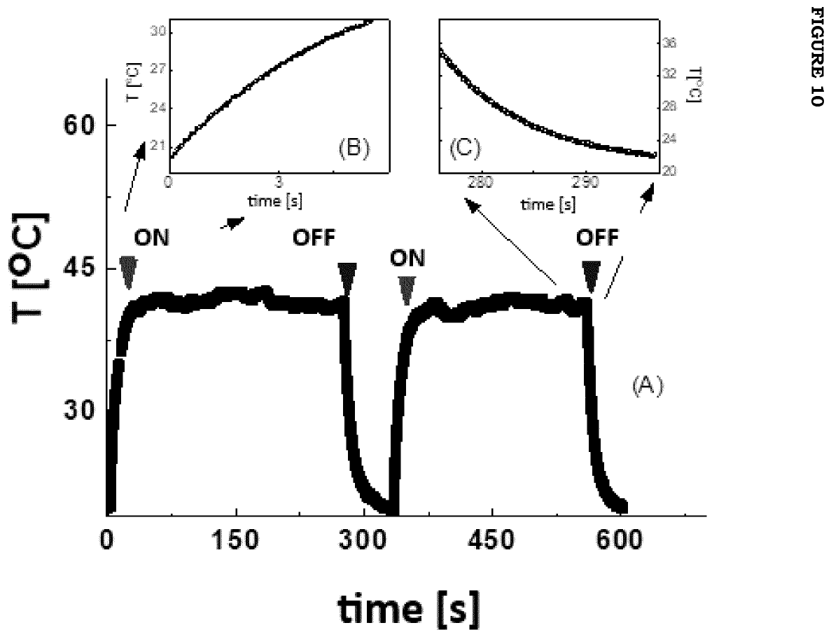

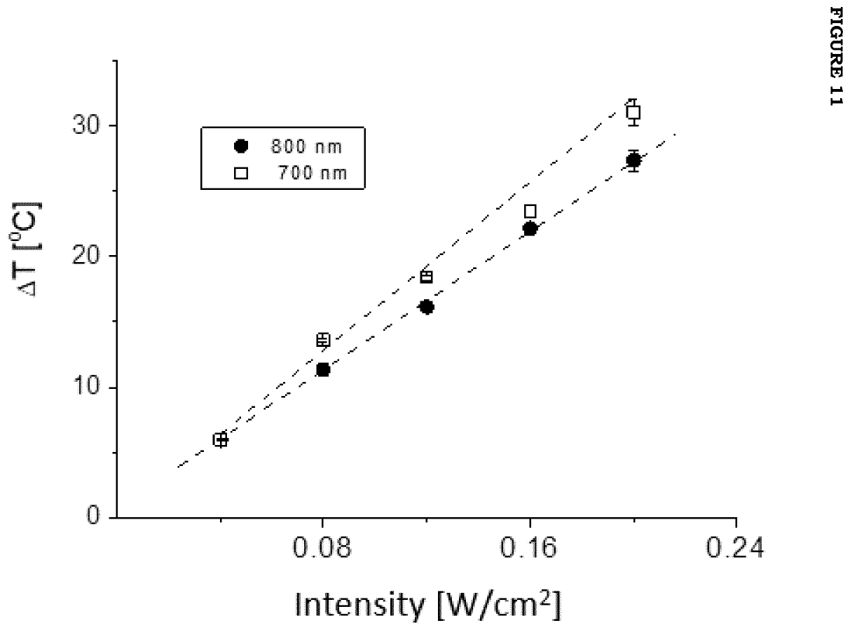

As a comparison, we show in FIGS. 10 and 11 the photo-thermal yields of the films prepared with PBNPs. A film prepared according to the formulation F5 (Tables 1,2,3) was continuously irradiated with radiation at wavelength of 0.80 μm (1=0.16 W/cm²), registering a photo-thermal response very similar to the one detected for the GNS films (formulations F1-F4). The temperature increase can be induced within a few seconds (mean rise time 5.8±0.5 s) and, once the radiation source has been turned off, the temperature of the film relaxes to room temperature within a few minutes (mean relaxation time equal to 8±0.5 s, FIG. 10), allowing to obtain activation and deactivation cycles of the photo-thermal effect for a very high number of times.

Also for the PBNP films, the photo-thermal effect broadly depends on the irradiation power (FIG. 11) and is well described by a direct proportionality relationship with a slope which is on the average higher than that found with the GNS nanoparticles, the concentration being equal (comparison between FIG. 9 and FIG. 11).

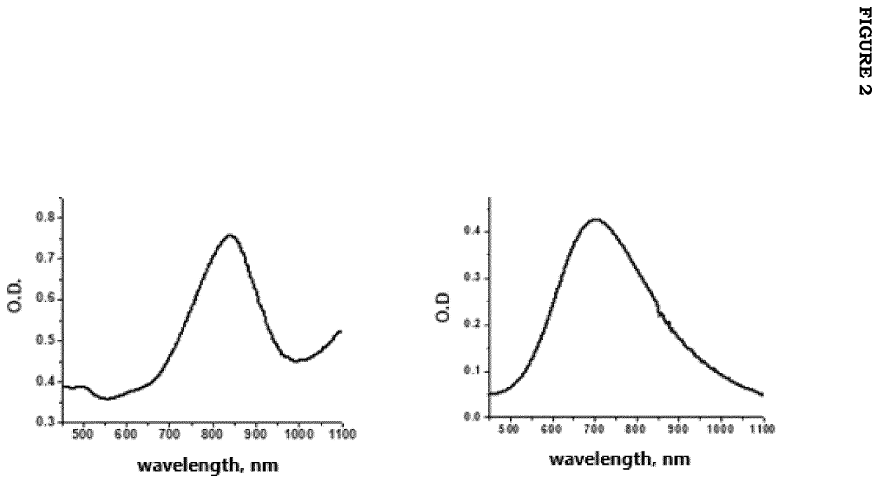

The film of formulation F5 has a high photo-thermal effect also under NIR irradiation at wavelength of 0.7 μm, (FIG. 11), with a slope about 17% higher than found for irradiation at wavelength of 0.80 μm (in accordance with the absorption spectrum of FIG. 2b).

### Example 5 Test of Photo-Thermal Efficiency In-Situ

### 5.A Test on Porcine Skin

The film of formulation F2, prepared with GNS nanoparticles (C=0.028 np/μm³) was layered on porcine skin and irradiated with NIR radiation at wavelength of 0.80 μm.

The film, of square shape and 2×2 cm² size, was placed on a portion of porcine skin ex-vivo (total thickness≈5 mm, of which at least the half consisting of subcutaneous fat, total mass=50 g). The irradiation spot had a 4 mm radius. We measured the temperature increase with a thermo-camera facing the intradermal side (on the opposite side of irradiation and of the applied film). The increase reached under continuous irradiation was ΔT=1.5° C. for a power P=100 mW (I=0.2 W/cm²) and ΔT=2.4° C. for a power P=200 mW (I=0.4 W/cm²).

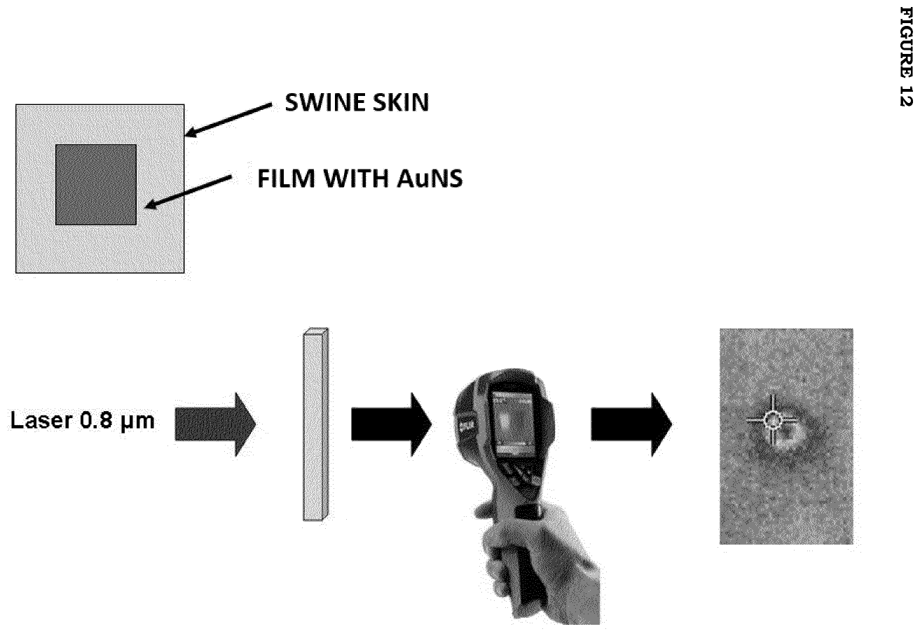

The control performed with a film of the same polymer composition but free of nanoparticles, shows instead a temperature increase lower than the sensitivity of the thermo-camera. The experiment is outlined in FIG. 12.

The temperature increase required for muscular thermal therapy is of about 2° C., compared to the temperature of the human body: therefore, the characterizations reported herein demonstrate the feasibility of the films developed for thermal therapy applications.

### 5.B Test on Human Body

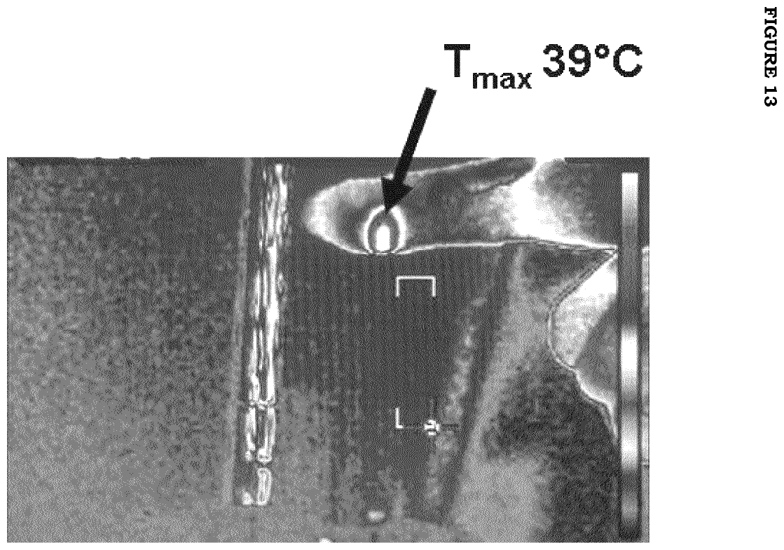

A film of formulation F2 (GNS nanoparticles, 2 cm×1 cm) was wrapped on the tip of a finger of one of the inventors. The film was irradiated with NIR radiation of wavelength of 0.80 μm and at power of 100 mW, continuously (1=0.16 W/cm²). The temperature increase measured by the thermo-camera at balance (reached after about 5 s) is reported in FIG. 13. The temperature measured at the center of the irradiated zone is 39° C., equal to an increase of about 4 Celsius degrees.

### Example 6. Optimized LED-Based Irradiation

The photothermal effect of the nanoparticle containing films described here can be activated by using low consumption infrared light emitting diodes (LED). The source we developed in this embodiment is a combination of light emitting diodes with a lens collimation setup. The source is controlled by a microcontroller and a temperature sensor.

### Light Emitting Diodes

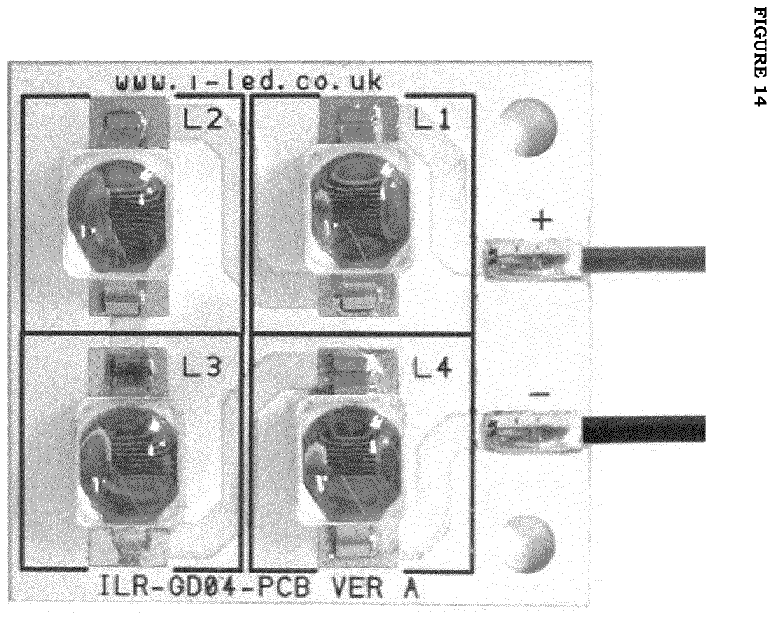

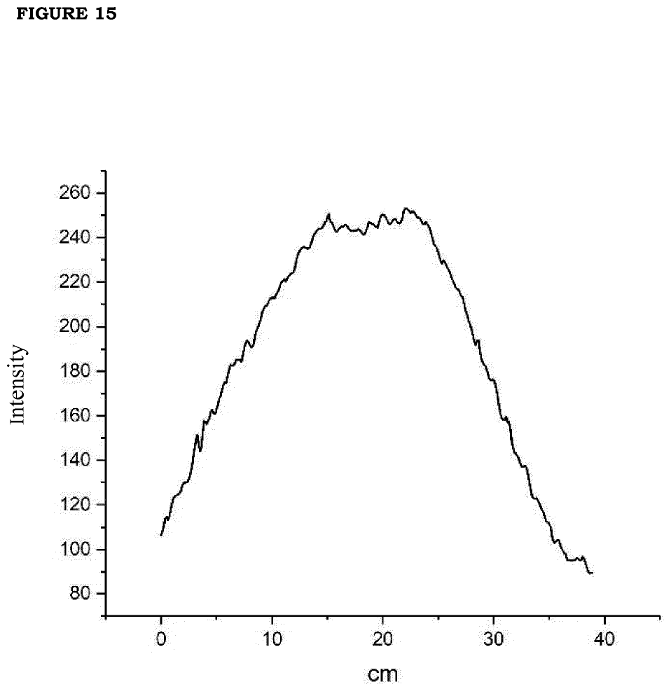

The LED source developed and applied for optimizing the photothermal applications of this invention consists of 4 LED matrices Dragon 4 IR. Each of them mounts 4 LED OSRAM IR Golden Dragon on an aluminum board. The scheme of single LED matrix equipped with 4 LED is displayed in FIG. 14. The emission spectrum of this LED source is tuned at wavelength around 850 nm. At these wavelengths in the Near Infrared Region the skin damage is limited to very high irradiance (see discussion below). Without any collection and field lens, the beam diameter at distance of 40 cm from source is 17 cm, while the beam diameter at 60 cm distance is 29 cm: the effective divergence angle is 24°±1°.The example of measured emission profile as a function of distance is shown in FIG. 15.

### Collimation Optics

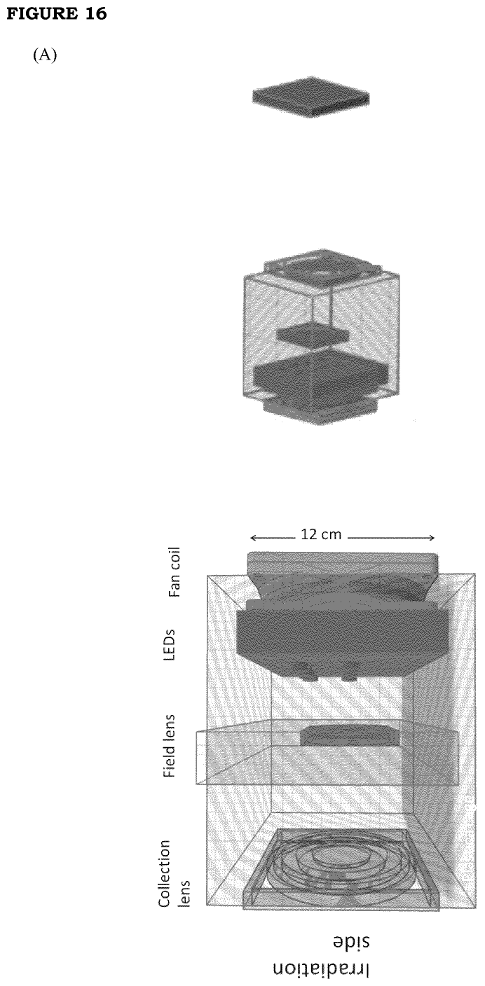

A Koheler illumination optical design is used. This allows to efficiently collect the NIR light and to deliver it on a defined area with an 10% illumination uniformity. This setup (FIGS. 16a, 16b and 16c) allows to reduce the heat losses and the dissipation into the environment and to have a perfect control of the size and shape of the illuminated region.

Possible choices of the set of lenses together with the main illumination features are reported in Table 5.

TABLE 5
Possible choices of lenses used in the LED source collection optics

| | f₁ | f₂ | q₂ | | FL size | Illumination |
|---|---|---|---|---|---|---|
| | [mm] | [mm] | [mm] | Magn. | [mm] | size [mm] |
| Cage 1 | 38 | 127 | 339 | 1.67 | 50.0 | 83.6 |
| Cage 2 | 66 | 127 | 249 | 0.96 | 76.2 | 73.3 |
| Cage 3 | 66 | 254 | 742 | 1.92 | 76.2 | 146.5 |
| Cage 4 | 50.8 | 127 | 286 | 1.25 | 63.5 | 79.4 |

Table 5. f₁, f₂ and q₂ are defined in FIG. 3C. Magn. is the magnification of the setup. FL size is the physical size of the field lens, the illumination size is the size of the illuminated area at the patient position (given by q₂).

The setup uses acrylic Fresnel lenses that can be easily shaped. Since the shape of the irradiation area is the shape of the field lens pupil magnified by the optical setup, it is possible to change the shape of the irradiation area to suit the particular need for the therapy.

The power of each LED spot using Fresnel lens was measured upon irradiation with maximum applied current (1.0 A) and the power values are reported in Table 6.

TABLE 6
Maximum power of Osram LED as a function of the
distance of the patch from the LED source. No
collimating lens setup was used in this case.

| Distance from LED | Maximum power of |
|---|---|
| source (cm) | each spot (mW) |
| 48 | 330 |
| 50 | 301 |
| 60 | 193 |

When the Koheler illumination setup is used to collect the NIR light the power stays constant with 10% when the observation plane is moved along the optical path by as much as 20 cm. This is due to the long Rayleigh range of the optical setup that we have built.

Compliance with the Skin Damage Threshold.

The directive of EU Parliament 2006/25/CE and regulation issued on 5 Apr. 2016 (regarding safety connected with the physical sources exploitation) suggests the following permitted levels of irradiation intensity (in case of exposure longer than 1 s) in the wavelength range 380-1200 nm:

I=2×10³C_{A} W/m²

with C_{A} given by:

C_{A}=10^{0.002(λ-700)}

When using a wavelength (λ) of about 850 nm (as in case of the LED source developed here) the maximum permitted irradiation intensity is:

I≅0.4 W/cm²

We used less than 0.3 W/cm², obtaining a photo-thermal effect sufficient for medical treatments. This makes our setup safe to be used on patients.

The working temperature of LED can reach 70° C. For this purpose, it is necessary to utilize a cooling fan that reduces the operating temperature of LED to about 33° C. The fan is driven by a 12 V of voltage providing 1.5 W, while the drive 1 A current of the LEDs is provided a 14V voltage power supply (15 W power).

### Control Electronics for the Source.

The electronic scheme with embedded LED is also equipped with thermometer Melexis MLX90614 allowing to control the temperature of patch through a hole in the Fresnel lenses. The LED and thermometer working conditions will be controlled by means of microcontroller STM32F072 Nucleo connected to PC. Moreover, the microcontroller will allow to monitor the temperature of the patch, to change the LED intensity and to activate or switch off the single LED matrix.

### Uniformity of Heating of the Patch.

The uniformity test, performed on a slab 15 cm in side, 2 mm thick sliced from the tread compound of tyres, chosen here as a test as a uniform absorber. Since the carbon black is uniformly dispersed in the sample, the measure of a constant temperature increase throughout the whole sample was taken as a measurement of the illumination uniformity.

TABLE 7
ΔT measured at different positions on the tyre slab. Full dimension
of the illuminated area = 120 mm × 120 mm

| Positions on the sample | ΔT | |
|---|---|---|
| 1 | 10.2° | C. |
| 2 | 10.9° | C. |
| 3 | 9.2° | C. |
| 4 | 7.0° | C. |
| 5 | 11.3° | C. |
| Max | 11.5° | C. |

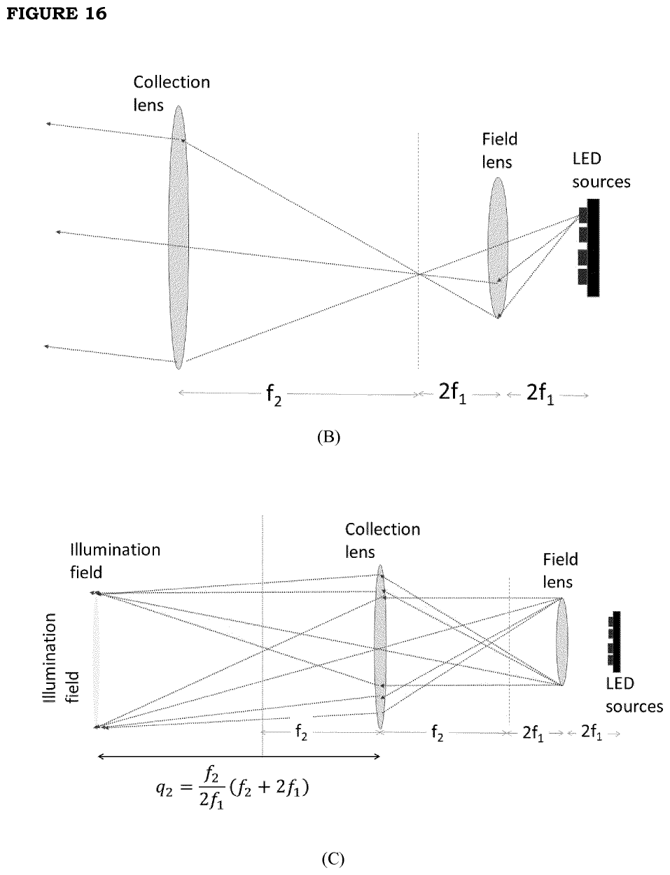

The position of the sampling points from which the results shown in the above table were obtained is shown in FIG. 17. The temperature increase is 10±1.3° C., with a minimum uniformity of 10.

### Photothermal Effect on Patches Irradiated by the LED Source.

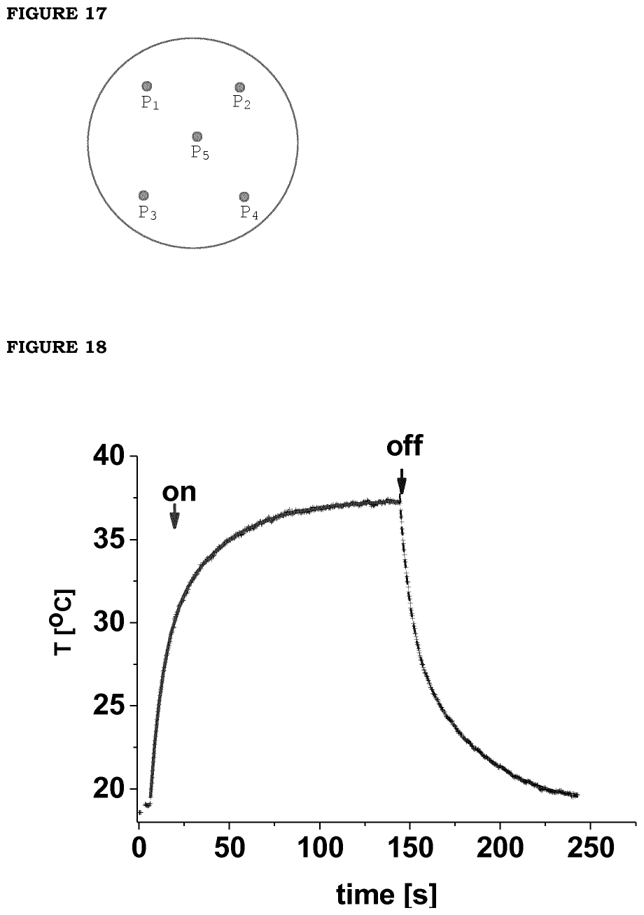

The photothermal efficiency of the patches prepared with PB nanoparticles was induced by irradiation with a 4 LED source (a single Dragon LED board) driven at the maximum current (I≅1 A). The temperature reaches a plateau value that corresponds to the increase ΔT=19±0.03° C. The temperature increases steadily and rapidly: within 10.1±0.1 s it reaches half the plateau value (FIG. 18). Similarly, when the LED source is switched off, the temperature decreases with a half decay time of 10.8±0.1° C.

1. Polymeric film containing nanoparticles, said nanoparticles being provided with a photo-thermal effect, which can be induced by irradiation with wavelength between 0.4 μm and 1.2 μm.

2. Polymeric film according to claim 1, wherein said nanoparticles are contained in the film or in part thereof, at a concentration between 0.005 and 0.1 nanoparticles/μm³.

3. Polymeric film according to claim 1, having thickness between 30 and 200 μm.

4. Polymeric film according to claim 1, wherein said nanoparticles have size between 5 and 100 nm.

5. Polymeric film according to claim 1, wherein said nanoparticles are selected from the group consisting of Gold Nanostars, pegylated Gold Nanostars, Prussian Blue nanoparticles and mixtures thereof.

6. Polymeric film according to claim 1, having specific absorption rate in the range of 30 [kW/g]≤SAR≤300 [kW/g].

7. Polymeric film according to claim 6, wherein said specific adsorption rate remains substantially constant during a working cycle comprising at least 40 irradiations.

8. Polymeric film according to claim 1, wherein the photo-thermal effect is obtained within 5 s from the beginning of said irradiation and ends within 10 s from the end of said irradiation.

9. Polymeric film according to claim 1, containing a polymer selected from the group consisting of polysaccharides, polylactides, polyacrylates, polymethacrylates, polyoleolefins, polyvinyl polymers, polyurethanes, polyamides, polyimides, polyethers, polyesters, polyacetates, polycarbonates, rubbers, polysiloxanes, cross-linked derivatives thereof and mixtures thereof.

10. Polymeric film according to claim 9, wherein said polymer is selected from the group consisting of polyvinyl alcohol, polyvinyl pyrrolidone, chitosan, and mixtures thereof, optionally cross-linked.

11. Process for preparing a polymeric film according to claim 1, comprising a step of adding a suspension containing said nanoparticles to the polymer making up said polymeric film or to a precursor thereof.

12. Process according to claim 11 further comprising a step of pegylating said particles and/or a step of cross-linking said polymer.

13. Process according to claim 12, wherein said step of cross-linking is carried out on the mixture resulting from the addition of the suspension, optionally pegylated.

14. Medical patch comprising a film as described in claim 1.

15. Method of thermal therapy comprising applying the medical patch according to claim 14 to a human or animal in need thereof.
